(19) United States
(12) Patent Application Publication
(10) Pub. No.: US 2007/0259952 A1
(43) Pub. Date: Nov. 8, 2007

(54) USES OF ESCITALOPRAM

(75) Inventor: Hans Torgny Svensson, Lidingo (SE)

Correspondence Address: DARBY & DARBY P.C., P.O. BOX 770, Church Street Station, New York, NY, 10008-0770, US

(73) Assignee: H. LUNDBECK A/S, Valby-Copenhagen (DK)

(21) Appl. No.: 11/741,371

(22) Filed: Apr. 27, 2007

Related U.S. Application Data
(60) Provisional application No. 60/746,238, filed on May 2, 2006.

(51) Int. Cl. A61K 31/343

(52) U.S. Cl. 514/469

(57) ABSTRACT

The present invention relates to the use of the compound escitalopram (INN-name), i.e. (S)-1-[3-(dimethylamino)propyl]-1-(4-fluorophenyl)-1,3-dihydro-5-isobenzofurancarbonitrile, or a pharmaceutically acceptable salt thereof for the preparation of a medicament for improving cognition in a condition where the cognitive processes are diminished.

### CROSS REFERENCE TO PRIOR APPLICATION

This application claims benefit under 35 U.S.C. 119(e) to U.S. Provisional Application No. 60/746238, filed May 2, 2006, which is incorporated by reference herein in its entirety.

### FIELD OF INVENTION

The present invention relates to the use of the compound escitalopram (INN-name), i.e. (S)-1-[3-(dimethylamino)propyl]-1-(4-fluorophenyl)-1,3-dihydro-5-isobenzofurancarbonitrile, or a pharmaceutically acceptable salt thereof for the preparation of a medicament for improving cognition in a condition where the cognitive processes are diminished.

### BACKGROUND OF THE INVENTION

Escitalopram is the (S)-enantiomer of the following structure:

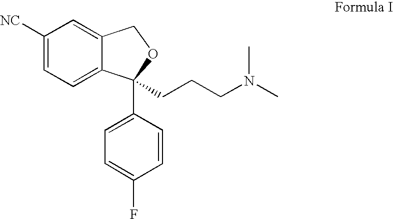

The compound and a method for its preparation are disclosed in U.S. Pat. No. 4,943,590. It is a selective, centrally acting serotonin (5-hydroxytryptamine; 5-HT) reuptake inhibitor, accordingly having antidepressant activities. The antidepressant activity and fast onset of action of the compound has e.g. been reported in Montgomery S A, et al Pharmacol Toxicol (2001) 88: 282-286.

Escitalopram is now marketed as an antidepressant and for the treatment of generalised anxiety disorder (GAD), panic disorder (PD) and social anxiety disorder (SAD).

WO0103694 discloses the use of escitalopram for the treatment of neurotic disorders.

Sanchez et al, 2004, Psychopharmacology, 174, 163-176, discloses data regarding the inhibition of the effects of escitalopram by R-citalopram and suggests that the better clinical effect observed for escitalopram versus citalopram is due to the removal of this inhibition.

Diminished cognitive processes can be experienced in several patient groups, e.g. by schizophrenic, depressive or psychotic patients and patients with attention deficit hyperactivity disorder (ADHD), Parkinson's disease, mild cognitive impairment (MCI), dementia, anxiety, age associated memory impairment, Alzheimer's Disease or post-traumatic stress disorder and patients taking benzodiazepines or tricyclic antidepressants and in a range of neurodegenerative diseases in addition to Parkinson's Disease and Alzheimer's Disease.

Diminished cognitive processes refer to the difficulties with attention, learning, memory and executive function (relevant reactions to external stimuli). These can include: deficits in attention, disorganized thinking, slow thinking, difficulty in understanding, poor concentration, impairment of problem solving, poor memory, difficulty in expressing thoughts and/or difficulty in integrating thoughts, feelings and behaviour and extinction of irrelevant thoughts as well as attention and vigilance, verbal learning and memory, visual learning and memory, speed of processing and social cognition.

Previous studies have shown that an increase in activity of dopamine neurons in the ventral tegmental area (VTA), in particular in burst firing, appear to selectively increase dopamine release in the medial prefrontal cortex. Dopamine in this brain region has been shown to play an important role in the modulation of some cognitive functions, e.g. working memory, through enhancement of glutamate function. Preclinical studies demonstrate that several SSRI's, i.e. fluoxetine, citalopram, paroxetine, sertraline and fluvoxamine all may cause a slight inhibition of the firing rate of mesocorticolimbic dopaminergic neurons in the VTA (Prisco and Eposito, 1995, Br J Pharmacol 116:1923-31; Di Masico et al, 1998, Brain Res Bull 46(6): 547-54, suggesting that these compounds cannot be used in improving cognitive processes. Moreover it has been reported that citalopram as adjunctive therapy to atypical antipsychotic treatment produces no significant cognitive improvement in patients with schizophrenia (Friedman et al, 2005, J. Clinical Psychopharmacology, 25(3), 237-42).

In contrast, the selective noradrenaline reuptake inhibitors (NRI), such as reboxetine enhances the excitability of dopaminergic neurons in the VTA with a preferential activation of their burst firing mode (Linner et al, 2001, JPET, 297(2):540-9) and administering of reboxetine has been suggested to have some beneficial effect in the treatment of ADHD.

The cognitive-enhancing effects of atypical, but not typical, antipsychotic drugs (APD's) have been ascribed to increased dopamine function and facilitation glutamatergic neurotransmission in the prefrontal cortical (as demonstrated by enhancement of NMDA receptor function; Ninan et al., 2003, Synapse, 48(2), 66-79).

Diminished cognitive processes in schizophrenia can to a certain extend be treated with atypical antipsychotics, however in general the effects of present antipsychotics on cognition are relatively modest, although the 2^{nd} generation (=atypical) antipsychotics have some effect (risperidone, olanzapine, clozapine, quetiapine, sertindole). Drugs approved for Alzheimer's dementia also have some effect on diminished cognitive processes, but work via completely different mechanisms, reflecting that cognition problems can have very different pathology, such as acethylcholinesterase inhibitors like donepezil, rivastigmine and galantamine. Memantine has weak NMDA antagonistic properties which gives a more “physiological” antagonism of too much background excitation, whereby it increases the signal to noise ratio on glutamate activity.

EP 474580 discloses that citalopram is useful in the treatment of cerebrovascular disorders and the brain damage and the impairment of memory functions in connection therewith, and that it shows inhibiting action on platelet aggregation. Furthermore EP 474580 suggests that citalopram has an anti-amnesic effect and improves cognitive function in elderly depressed patients having concomitant dementia. It is mentioned that the optical isomers of the compounds may also be used. The effect mentioned in this disclosure suggests a neuroprotective role.

Accordingly there is a significant unmet medical need for therapies, which can improve the cognitive symptoms in various diseases and disorders and/or have lowered side effects as compared to known treatments of cognitive symptoms.

Escitalopram has now been found to increase the firing rate of dopamine cells in the VTA, to stimulate the glutamate-driven burst firing in VTA and to facilitate NMDA-induced currents.

### SUMMARY OF THE INVENTION

In a first aspect the present invention relates to the use of escitalopram or a salt thereof for the preparation of a medicament for improving cognition in a condition where the cognitive processes are diminished.

In a second aspect the present invention relates the use of escitalopram or a pharmaceutically acceptable salt thereof in combination with one or more antipsychotic agents for the preparation of a medicament for improving cognition in a condition where the cognitive processes are diminished.

In a third aspect the present invention relates the use of escitalopram or a pharmaceutically acceptable salt thereof for the preparation of a medicament to be used in the adjunct treatment with an antipsychotic agent for improving cognition in a condition where the cognitive processes are diminished.

In a fourth aspect the present invention relates to a pharmaceutical composition comprising escitalopram or a pharmaceutically acceptable salt thereof and one or more antipsychotic compounds for improving cognition in a condition where the cognitive processes are diminished.

In a sixth aspect the present invention relates to a method of improving cognition in a condition where the cognitive processes are diminished comprising administering escitalopram or a pharmaceutically acceptable salt thereof.

### BRIEF DESCRIPTION OF THE DRAWINGS

FIG. 1: Escitalopram increases the firing rate of dopamine cells in the VTA in vivo and R-citalopram blocks the effect of escitalopram.

FIG. 2: Escitalopram stimulates the glutamate-driven burst firing in VTA dopamine cells in vivo more potently than citalopram and R-citalopram blocks the effect of escitalopram.

FIG. 3: Effect of risperidone (atypical antipsychotic), citalopram, escitalopram, and reboxetine (NRI) on NMDA-induced currents in pyramidal cells of the medial prefrontal cortex in vitro.

### DETAILED DESCRIPTION OF THE INVENTION

According to the present invention, a novel use of escitalopram or a pharmaceutically acceptable salt thereof, namely for the preparation of a medicament for improving cognition in a condition where the cognitive processes are diminished is provided.

It has now been found that escitalopram increases the firing rate of dopamine cells in the VTA in vivo, an effect blocked by R-citalopram. Additionally it has been found escitalopram compared with citalopram more potently and also dose-dependently stimulates the glutamate-driven burst firing in VTA dopamine cells in vivo, an effect that is blocked by R-citalopram. Moreover it has been found that escitalopram and reboxetine but not citalopram facilitate NMDA-induced currents in pyramidal cells of the medial prefrontal cortex in vitro.

The present findings indicate that escitalopram in similarity with NRIs, such as reboxetine, enhance the excitability of the dopaminergic system. Additionally the present findings indicate that escitalopram in similarity with atypical, but not typical antipsychotic drugs, as well as reboxetine facilitates prefrontal glutaminergic neurotransmission.

These effects strongly suggest that escitalopram can be used for improving cognition in a condition where the cognitive processes are diminished as well as augmenting the response to exposure therapy.

Additionally the present findings also strongly suggest escitalopram may be used as adjunct treatment to typical antipsychotic drugs for improving cognition in a condition where the cognitive processes are diminished and in the treatment of schizophrenia and other related diseases and disorders.

The phrase “diminished cognitive processes” refers to the difficulties with attention, learning, memory and executive function (relevant reactions to external stimuli). These can include: deficits in attention, disorganized thinking, slow thinking, difficulty in understanding, poor concentration, impairment of problem solving, poor memory, difficulty in expressing thoughts and/or difficulty in integrating thoughts, feelings and behaviour and extinction of irrelevant thoughts as well as attention and vigilance, verbal learning and memory, visual learning and memory, speed of processing and social cognition.

One embodiment of the present invention relates to the use of escitalopram or a pharmaceutically acceptable salt thereof for the preparation of a medicament for improving cognition in a condition where the cognitive processes are diminished wherein the condition is associated with schizophrenia. In another embodiment of the invention the condition is associated with Parkinson's Disease. In another embodiment of the invention the condition is associated with dementia, such as AIDS dementia. In another embodiment of the invention the condition is associated with an anxiety disorder. In another embodiment of the invention the condition is associated with age associated memory impairment. In another embodiment of the invention the condition is associated with depression, including major depression, in particular in elderly. In another embodiment of the invention the condition is associated with the use of benzodiazepines. In another embodiment of the invention the condition is associated with the use of tricyclic antidepressants. In another embodiment of the invention the condition is associated with Alzheimer's Disease. In another embodiment of the invention the condition is associated with attention deficit hyperactivity disorder (ADHD). In another embodiment of the invention the condition is associated with post-traumatic stress disorder (PTSD).

In a further embodiment of the invention the medicament is for administration as a unit dose. In further embodiment of the invention the unit dose is containing escitalopram in an amount from 1.0 mg to 50 mg, more preferred 5 mg/day to 20 mg/day, most preferably 10 mg. In a further embodiment the unit dose is given once daily. In a further embodiment the daily doses are, 5, 10 or 20 mg.

Another embodiment of the present invention relates to the use of escitalopram or a pharmaceutically acceptable salt thereof in combination with one or more antipsychotic compounds for the preparation of a medicament for improving cognition in a condition where the cognitive processes are diminished wherein the condition is associated with schizophrenia. In another embodiment of the invention the condition is associated with Parkinson's Disease. In another embodiment of the invention the condition is associated with dementia, such as AIDS dementia. In another embodiment of the invention the condition is associated with an anxiety disorder. In another embodiment of the invention the condition is associated with age associated memory impairment. In another embodiment of the invention the condition is associated with depression, including major depression, in particular in elderly. In another embodiment of the invention the condition is associated with the use of benzodiazepines. In another embodiment of the invention the condition is associated with the use of tricyclic antidepressants. In another embodiment of the invention the condition is associated with Alzheimer's Disease. In another embodiment of the invention the condition is associated with attention deficit hyperactivity disorder (ADHD). In another embodiment of the invention the condition is associated with post-traumatic stress disorder (PTSD). In another embodiment of the invention the antipsychotic compound is a compound approved for the treatment of a psychotic condition. In a further embodiment of the invention the antipsychotic compound is selected from the list of Asenapine, Blonanserin, Iloperidone, Paliperidone, Bifeprunox, Lurasidone, Ocaperidone, Talnetant, ACP 104, SLV 310, ACR 16, YKP 1358, GW 773812, RGH 188, SLV 314, Y-931, BL 1020, Chlorpromazine, Levomepromazine, Promazine, Acepromazine, Triflupromazine, Cyamemazine, Chlorproethazine, Dixyrazine, Fluphenazine, Perphenazine, Prochlorperazine, Thiopropazate, Trifluoperazine, Acetophenazine, Thioproperazine, Butaperazine, Perazine, Periciazine, Thioridazine, Mesoridazine, Pipotiazine, Haloperidol, Trifluperidol, Melperone, Moperone, Pipamperone, Bromperidol, Benperidol, Droperidol, Fluanisone, Oxypertine, Molindone, Sertindole, Ziprasidone, Flupentixol, Clopenthixol, Chlorprothixene, Tiotixene, Zuclopenthixol, Fluspirilene, Pimozide, Penfluridol, Loxapine, Clozapine, Olanzapine, Quetiapine, Sulpiride, Sultopride, Tiapride, Remoxipride, Amisulpride, Veralipride, Levosulpiride, Prothipendyl, Risperidone, Clotiapine, Mosapramine, Zotepine and Aripiprazole.

A further embodiment of the present invention relates to the use of escitalopram or a pharmaceutically acceptable salt thereof for the preparation of a medicament to be used in the adjunct treatment with an antipsychotic compound for improving cognition in a condition where the cognitive processes are diminished wherein the condition is associated with schizophrenia. In another embodiment of the invention the condition is associated with Parkinson's Disease. In another embodiment of the invention the condition is associated with dementia, such as AIDS dementia. In another embodiment of the invention the condition is associated with an anxiety disorder. In another embodiment of the invention the condition is associated with age associated memory impairment. In another embodiment of the invention the condition is associated with depression, including major depression, in particular in elderly. In another embodiment of the invention the condition is associated with the use of benzodiazepines. In another embodiment of the invention the condition is associated with the use of tricyclic antidepressants. In another embodiment of the invention the condition is associated with Alzheimer's Disease. In another embodiment of the invention the condition is associated with attention deficit hyperactivity disorder (ADHD). In another embodiment of the invention the condition is associated with post-traumatic stress disorder (PTSD). In another embodiment of the invention the antipsychotic compound is a compound approved for the treatment of a psychotic condition. In a further embodiment of the invention the antipsychotic agent is selected from the list of Asenapine, Blonanserin, Iloperidone, Paliperidone, Bifeprunox, Lurasidone, Ocaperidone, Talnetant, ACP 104, SLV 310, ACR 16, YKP 1358, GW 773812, RGH 188, SLV 314, Y-931, BL 1020, Chlorpromazine, Levomepromazine, Promazine, Acepromazine, Triflupromazine, Cyamemazine, Chlorproethazine, Dixyrazine, Fluphenazine, Perphenazine, Prochlorperazine, Thiopropazate, Trifluoperazine, Acetophenazine, Thioproperazine, Butaperazine, Perazine, Periciazine, Thioridazine, Mesoridazine, Pipotiazine, Haloperidol, Trifluperidol, Melperone, Moperone, Pipamperone, Bromperidol, Benperidol, Droperidol, Fluanisone, Oxypertine, Molindone, Sertindole, Ziprasidone, Flupentixol, Clopenthixol, Chlorprothixene, Tiotixene, Zuclopenthixol, Fluspirilene, Pimozide, Penfluridol, Loxapine, Clozapine, Olanzapine, Quetiapine, Sulpiride, Sultopride, Tiapride, Remoxipride, Amisulpride, Veralipride, Levosulpiride, Prothipendyl, Risperidone, Clotiapine, Mosapramine, Zotepine and Aripiprazole.

A further embodiment of the present invention relates to a method of improving cognition in a condition where the cognitive processes are diminished comprising administering escitalopram or a pharmaceutically acceptable salt thereof wherein the condition is associated with schizophrenia. In another embodiment of the invention the condition is associated with Parkinson's Disease. In another embodiment of the invention the condition is associated with dementia, such as AIDS dementia. In another embodiment of the invention the condition is associated with an anxiety disorder. In another embodiment of the invention the condition is associated with age associated memory impairment. In another embodiment of the invention the condition is associated with depression, including major depression, in particular in elderly. In another embodiment of the invention the condition is associated with the use of benzodiazepines. In another embodiment of the invention the condition is associated with the use of tricyclic antidepressants. In another embodiment of the invention the condition is associated with Alzheimer's Disease. In another embodiment of the invention the condition is associated with attention deficit hyperactivity disorder (ADHD). In another embodiment of the invention the condition is associated with post-traumatic stress disorder (PTSD).

A further embodiment of the present invention relates to the use of escitalopram for the preparation of a medicament for the treatment of schizophrenia wherein the negative and cognitive symptoms are improved.

A further embodiment of the present invention relates to the use of escitalopram for the preparation of a medicament for the treatment of attention deficit hyperactivity disorder (ADHD).

Increasing glutamatergic neurotransmission pharmacologically have been shown to be usefull in enhancing the effect of exposure-based therapy which is likely transferable to other cognitive behavioural therapies (Augmentation of exposure therapy with D-cycloserine for social anxiety disorder, Arch Gen Psychiatry, vol 63, 298-304; Pharmacological Treatments that Facilitate Extinction of Fear: Relevance to Psychotherapy, The journal of American society for experimental neurotherapeutics, vol. 3, 82-96; Ressler et al., Arch Gen Psychiatry, 2004, 61(11):1136-44. Cognitive enhancers as adjuncts to psychotherapy: use of D-cycloserine in phobic individuals to facilitate extinction of fear). Thus for escitalopram, the facilitation of the glutamatergic neurotransmission in prefrontal cortex, likely means that escitalopram can be used to enhance the effect of psychotherapy, e.g. exposure-based therapies. Escitalopram could affect anxiety disorders, both directly through the SRI component and through facilitation of e.g. extinction processes during psychotherapy.

Thus a further embodiment of the present invention relates to the use of escitalopram for the preparation of a medicament useful in facilitating psychotherapy, cognitive behavioural therapy and exposure-based therapy.

The present invention thus provides a significant improvement in the treatment of cognition in conditions where the cognitive processes are diminished. The advantages of the invention, compared to known treatments are better efficacy and potentionally lowered side-effects. Moreover some patients groups that could not be treated at all before might, by the use of escitalopram according to the present invention, experience improved effects on cognitive processes.

According to the invention, escitalopram or a pharmaceutically acceptable salt thereof may be administered in any suitable way e.g. orally or parenterally, and it may be presented in any suitable form for such administration, e.g. in the form of tablets, capsules, powders, syrups or solutions or dispersions for injection. Preferably, and in accordance with the purpose of the present invention, the compound of the invention is administered in the form of a solid pharmaceutical entity, suitably as a tablet or a capsule or in the form of a suspension, solution or dispersion for injection.

Methods for the preparation of solid pharmaceutical preparations are well known in the art. Tablets may thus be prepared by mixing the active ingredients with ordinary adjuvants and/or diluents and subsequently compressing the mixture in a convenient tabletting machine. Examples of adjuvants or diluents comprise: corn starch, lactose, talcum, magnesium stearate, gelatine, lactose, gums, and the like. Any other adjuvant or additive such as colourings, flavourings, preservatives, etc. may also be used provided that they are compatible with the active ingredients.

Escitalopram is preferably used in the form of the oxalate salt thereof, escitalopram as the base or as the HBr salt or the likes or any other pharmaceutically acceptable salt thereof could also be used.

Escitalopram is most conveniently administered orally in unit dosage forms such as tablets or capsules, containing the active ingredient in a dose from about 1.0 mg to 50 mg, more preferred 5 mg/day to 20 mg/day, most preferably 10 mg. Such a unit dose is preferably given once daily. Preferred daily doses are, 5, 10 or 20 mg.

The oxalate of escitalopram may be prepared as described in U.S. Pat. No. 4,943,590 and the base and other pharmaceutically acceptable salts may be obtained from the base by standard procedures.

Thus the acid addition salts used according to the invention may be obtained by treatment of escitalopram with the acid in an inert solvent followed by precipitation, isolation and optionally re-crystallisation by known methods and if desired micronisation of the crystalline product by wet or dry milling or another convenient process, or preparation of particles from a solvent-emulsification process.

The antipsychotic compounds that can be used according to the present invention is preferably selected from the list of Asenapine, Blonanserin, Iloperidone, Paliperidone, Bifeprunox, Lurasidone, Ocaperidone, Talnetant, ACP 104, SLV 310, ACR 16, YKP 1358, GW 773812, RGH 188, SLV 314, Y-931, BL 1020, Chlorpromazine, Levomepromazine, Promazine, Acepromazine, Triflupromazine, Cyamemazine, Chlorproethazine, Dixyrazine, Fluphenazine, Perphenazine, Prochlorperazine, Thiopropazate, Trifluoperazine, Acetophenazine, Thioproperazine, Butaperazine, Perazine, Periciazine, Thioridazine, Mesoridazine, Pipotiazine, Haloperidol, Trifluperidol, Melperone, Moperone, Pipamperone, Bromperidol, Benperidol, Droperidol, Fluanisone, Oxypertine, Molindone, Sertindole, Ziprasidone, Flupentixol, Clopenthixol, Chlorprothixene, Tiotixene, Zuclopenthixol, Fluspirilene, Pimozide, Penfluridol, Loxapine, Clozapine, Olanzapine, Quetiapine, Sulpiride, Sultopride, Tiapride, Remoxipride, Amisulpride, Veralipride, Levosulpiride, Prothipendyl, Risperidone, Clotiapine, Mosapramine, Zotepine and Aripiprazole. In particular, Risperidone, Olanzapine, Clozapine, Quetiapine, Sertindole, Ocaperidone, Iloperidone, BL 1020, YKPI358, Ziprasidone, Paliperidone, Asenopine, Lurasidone, Aripripazol and Bifeprunox. According to the present invention these antipsychotic compounds should be administered in their respectively clinically approved doses, such as approved by the FDA and/or EMEA. However, it will be understood that the amount of the above-mentioned compounds actually administered will be determined by a physician, according to the relevant circumstances, including the condition to be treated, the chosen route of administration, the actual compound administered, the age, weight, and response of the individual patient, and the severity of the patient's symptoms.

Experimentals

In vivo assessment of dopamine neuron firing in rat ventral tegmental area was conducted as described by Schilstrom et al. (Neuroscience 125:957-64, 2004). Briefly, male Sprague Dawley rats (250-300 g) were anesthetized with chloral hydrate (400 mg/kg i.p.) and mounted in a stereotactic frame. Anesthesia was maintained throughout the experiment with periodical injections of chloral hydrate and body temperature was kept at 37° C. with a rectal thermometer connected to an electrical heating pad. Recording electrodes were pulled in a Narishige vertical puller from borosilicate glass capillaries with outer and inner diameters of 1.50 and 1.17 mm, respectively, and filled with 2% Pontamine Sky Blue in 2 M NaCl. The electrodes were broken under microscope to yield an impedance of 2-4 MΩ at 135 Hz and then slowly lowered into a hole drilled in the skull above the VTA (3.2±0.3 mm anterior of the interaural line and 0.7±0.2 mm lateral to the midline by using a hydraulic microdrive. Extracellular electrical activity was amplified, filtered (band pass 0.3-3 kHz), discriminated and monitored on an oscilloscope and an audiomonitor. Discriminated spikes were fed via a CED 1401 interface to a computer running CED Spike 2 software. After the experiment the recording site was marked by iontophoresis of Pontamine Sky Blue into the tissue. The rat was killed with an overdose of anesthetic, and the brain was removed and placed in 25% sucrose 10% paraformaldehyde solution. Sections of the VTA were cut (50 μM) and stained with Neutral Red and the recording sites were verified by light microscopy.

In vitro studies of facilitation of NMDA-induced currents in pyramidal cells of the medial prefrontal cortex were conducted as described by Jardemark et al. (Int J Neuropsychopharmacol. 8: 157-162, 2005). Male Sprague-Dawley rats housed under standard laboratory conditions were used. Brain slices were prepared in the following manner: Rats were decapitated under halothane anaesthesia and the brains were rapidly removed and cooled in ice-cold Ringer's solution. The brains were then cut coronally in order to produce 450 μm slices. The brain slices were kept submerged in aerated Ringer's solution at room temperature for at least 1 hour to allow for recovery. Subsequently a single slice containing mPFC was then transferred to a recording chamber (32° C.), in which it was held submerged in-between two nylon nets. The chamber was continuously perfused by aerated Ringer's solution at a flow rate of 2 ml/min. Standard intracellular and single-electrode voltage-clamp techniques were used to record pyramidal cells in layers V and VI of the mPFC in slice preparations as described previously. Briefly, electrodes were pulled from borosilicate glass capillaries by using a horizontal electrode puller. Recording electrodes were filled with 2 M Potassium Acetate (the tip resistance was between 20 and 160 MΩ) and used for recording the mPFC neurons with an Axoclamp 2A amplifier. Single electrode voltage-clamp (holding potential −60 mV) was performed in the discontinuous mode with a sampling rate of 5-6.2 kHz. The voltage recordings were acquired using a digital/analogue sampling and acquisition software. During the voltage-clamp recordings of the NMDA-evoked currents tetrodotoxin (TTX; 0.5 μM to block the action potentials), glycine (1 μM, to enhance the NMDA-induced responses) bicucculine (5 μM, to block the γ-aminobutyric acid type A (GABA_{A}) responses) were routinely included in Ringer's solution. NMDA (5-25 μM was also applied via bath perfusion of Ringer's solution to induce inward currents. The effects of the drugs or drug combinations on the NMDA-induced currents were calculated by dividing the NMDA-induced currents after the bath application of the drugs by the control NMDA-induced currents. The results shown in the figure are presented as mean ± S.E.M.

Description of the Results:

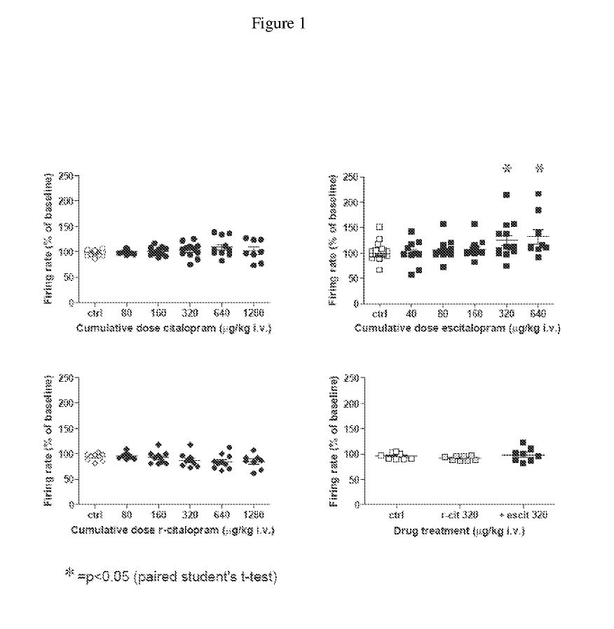

FIG. 1 describes the effect of citalopram and each of its enantiomers on firing rate of dopamine cells in the VTA. It is shown that citalopram at the tested doses (0-1280 μg/kg) does not increase the firing rate. On the other hand escitalopram elevates firing rate statistically significant at 320 μg/kg and this effect appears to be maintained at the higher dose (640 μg/kg). Furthermore it is shown that the firing rate is unaffected by R-citalopram and that the effect of escitalopram (320 μg/kg) can be blocked by pre-treatment with 320 μg/kg of R-citalopram, thus further substantiating that this effect is specific to escitalopram.

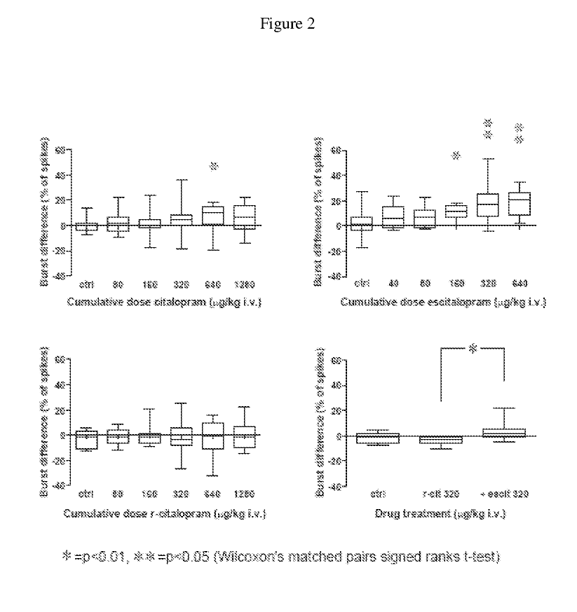

FIG. 2 describes the effect of citalopram and each of its enantiomers on burst firing of the dopaminergic cells recorded in the in the VTA. It was shown that escitalopram increases burst firing statistically significant dose-dependently at doses equal to and above 160 μg/kg. At the highest dose tested (640 μg/kg) escitalopram causes a burst difference of about 20%. Citalopram also appear to increase burst firing, allthough this only reaches significance at the 640 μg/kg dose, and the maximal response appears to be approximately 10% burst difference. Further it is shown that R-citalopram alone has no effect on burst firing and that R-citalopram (320 μg/kg) significantly inhibits the effect of escitalopram (320 μg/kg).

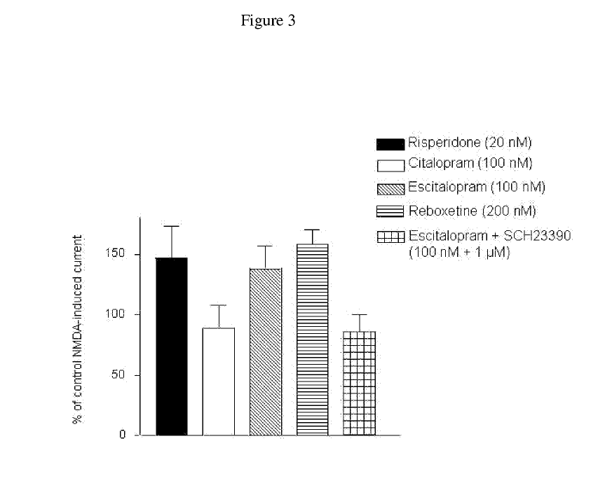

FIG. 3 describes the effect of risperidone (atypical antipsychotic), citalopram, escitalopram and reboxetine (NRI) on NMDA-induced currents in pyramidal cells of the prefrontal cortex. It has previously been shown that atypical antipsychotics increase NMDA-induced currents in this preparation. This is examplified by risperidone. It is shown that reboxetine (NRI) but not citalopram (SSRI), nor fluoxetine (data not shown) increases NMDA-induced currents. Surprisingly escitalopram facilitates the NMDA-induced currents to similar extent as the risperidone and reboxetine. It is furthermore shown that this effect is likely mediated through the dopamine D1 receptor as a dopamine D1 receptor antagonist (SCH 23390) reverses the effect of escitalopram.

1. A method of improving cognition in a condition where the cognitive processes are diminished comprising administering a therapeutically effective amount of escitalopram or a pharmaceutically acceptable salt thereof.

2. The method according to claim 1 wherein the condition is associated with schizophrenia.

3. The method according to claim 1 wherein the condition is associated with Parkinson's Disease.

4. The method according to claim 1 wherein the condition is associated with dementia.

5. The method according to claim 1 wherein the condition is associated with an anxiety disorder.

6. The method according to claim 1 wherein the condition is associated with age associated memory impairment.

7. The method according to claim 1 wherein the condition is associated with depression.

8. The method according to claim 1 wherein the condition is associated with the use of benzodiazepines.

9. The method according to claim 1 wherein the condition is associated with the use of tricyclic antidepressants.

10. The method according to claim 1 wherein the condition is associated with Alzheimer's Disease.

11. The method according to claim 1 wherein the condition is associated with attention deficit hyperactivity disorder (ADHD).

12. The method according to claim 1 wherein the condition is associated with post-traumatic stress disorder (PTSD).

13. The use method according to claim 1 wherein the escitalopram or a pharmaceutically acceptable salt thereof is administered as a unit dose.

14. The use method according to claim 13 wherein the unit dose contains 1.0 mg to 50 mg of escitalopram or a pharmaceutically acceptable salt thereof.

15. The method accordinh to claim 13 wherein the unit dose is given once daily.

16. The method according to claim 15 wherein the daily doses are 5, 10 or 20 mg.

17. A method of improving cognition in a condition where the cognitive processes are diminished comprising administering a therapeutically effective amount of escitalopram or a pharmaceutically acceptable salt thereof in combination with one or more antipsychotic compounds.

18. The method according to claim 17 wherein the condition is associated with schizophrenia.

19. The method according to claim 17 wherein the condition is associated with Parkinson's Disease.

20. The method according to claim 17 wherein the condition is associated with dementia.

21. The method according to claim 17 wherein the condition is associated with an anxiety disorder.

22. The method according to claim 17 wherein the condition is associated with age associated memory impairment.

23. The method according to claim 17 wherein the condition is associated with depression.

24. The method according to claim 17 wherein the condition is associated with the use of benzodiazepines.

25. The method according to claim 17 wherein the condition is associated with the use of tricyclic antidepressants.

26. The method according to claim 17 wherein the condition is associated with Alzheimer's Disease.

27. The method according to claim 17 wherein the condition is associated with attention deficit hyperactivity disorder (ADHD).

28. The method according to claim 17 wherein the condition is associated with post-traumatic stress disorder (PTSD).

29. The method according to claim 17 wherein the antipsychotic agent is an agent approved for the treatment of a psychotic condition.

30. The method according to claim 17 wherein the antipsychotic compound is selected from Asenapine, Blonanserin, Iloperidone, Paliperidone, Bifeprunox, Lurasidone, Ocaperidone, Talnetant, ACP 104, SLV 310, ACR 16, YKP 1358, GW 773812, RGH 188, SLV 314, Y-931, BL 1020, Chlorpromazine, Levomepromazine, Promazine, Acepromazine, Triflupromazine, Cyamemazine, Chlorproethazine, Dixyrazine, Fluphenazine, Perphenazine, Prochlorperazine, Thiopropazate, Trifluoperazine, Acetophenazine, Thioproperazine, Butaperazine, Perazine, Periciazine, Thioridazine, Mesoridazine, Pipotiazine, Haloperidol, Trifluperidol, Melperone, Moperone, Pipamperone, Bromperidol, Benperidol, Droperidol, Fluanisone, Oxypertine, Molindone, Sertindole, Ziprasidone, Flupentixol, Clopenthixol, Chlorprothixene, Tiotixene, Zuclopenthixol, Fluspirilene, Pimozide, Penfluridol, Loxapine, Clozapine, Olanzapine, Quetiapine, Sulpiride, Sultopride, Tiapride, Remoxipride, Amisulpride, Veralipride, Levosulpiride, Prothipendyl, Risperidone, Clotiapine, Mosapramine, Zotepine and Aripiprazole.

31. A method of improving cognition in a condition where the cognitive processes are diminished comprising administering a therapeutically effective amount of escitalopram or a pharmaceutically acceptable salt thereof in adjunct treatment with an antipsychotic agent.

32. The method according to claim 31 wherein the condition is associated with schizophrenia.

33. The method according to claim 31 wherein the condition is associated with Parkinson's Disease.

34. The method according to claim 31 wherein the condition is associated with dementia.

35. The method according to claim 31 wherein the condition is associated with an anxiety disorder.

36. The method according to claim 31 wherein the condition is associated with age associated memory impairment.

37. The method according to claim 31 wherein the condition is associated with depression.

38. The method according to claim 31 wherein the condition is associated with the use of benzodiazepines.

39. The method according to claim 31 wherein the condition is associated with the use of tricyclic antidepressants.

40. The method according to claim 31 wherein the condition is associated with Alzheimer's Disease.

41. The method according to claim 31 wherein the condition is associated with attention deficit hyperactivity disorder (ADHD).

42. The method according to claim 31 wherein the condition is associated with post-traumatic stress disorder (PTSD).

43. The method according to claim 31 wherein the antipsychotic agent is an agent approved for the treatment of a psychotic condition.

44. The method according to claim 31 wherein the antipsychotic agent is selected from Asenapine, Blonanserin, Iloperidone, Paliperidone, Bifeprunox, Lurasidone, Ocaperidone, Talnetant, ACP 104, SLV 310, ACR 16, YKP 1358, GW 773812, RGH 188, SLV 314, Y-931, BL 1020, Chlorpromazine, Levomepromazine, Promazine, Acepromazine, Triflupromazine, Cyamemazine, Chlorproethazine, Dixyrazine, Fluphenazine, Perphenazine, Prochlorperazine, Thiopropazate, Trifluoperazine, Acetophenazine, Thioproperazine, Butaperazine, Perazine, Periciazine, Thioridazine, Mesoridazine, Pipotiazine, Haloperidol, Trifluperidol, Melperone, Moperone, Pipamperone, Bromperidol, Benperidol, Droperidol, Fluanisone, Oxypertine, Molindone, Sertindole, Ziprasidone, Flupentixol, Clopenthixol, Chlorprothixene, Tiotixene, Zuclopenthixol, Fluspirilene, Pimozide, Penfluridol, Loxapine, Clozapine, Olanzapine, Quetiapine, Sulpiride, Sultopride, Tiapride, Remoxipride, Amisulpride, Veralipride, Levosulpiride, Prothipendyl, Risperidone, Clotiapine, Mosapramine, Zotepine and Aripiprazole.

45. A pharmaceutical composition comprising escitalopram or a pharmaceutically acceptable salt thereof and one or more antipsychotic compounds for improving cognition in a condition where the cognitive processes are diminished.

46.-57. (canceled)

58. A method of treating schizophrenia wherein the negative and cognitive symptoms are improved comprising administering a therapeutically effective amount of escitalopram or a pharmaceutically acceptable salt thereof.

59. The method according to claim 58 wherein the escitalopram or a pharmaceutically acceptable salt thereof is as a unit dose.

60. The method according to claim 59 wherein the unit dose contains 1.0 mg to 50 mg of escitalopram or a pharmaceutically acceptable salt thereof.

61. The method according to claim 59 wherein the unit dose is given once daily.

62. The method according to claim 61 wherein the daily doses are 5, 10 or 20 mg.

63. A method of treating attention deficit hyperactivity disorder (ADHD) comprising administering a therapeutically effective amount of escitalopram or a pharmaceutically acceptable salt thereof.

64. The method according to claim 63 wherein the escitalopram or a pharmaceutically acceptable salt thereof is administered as a unit dose.

65. The method according to claim 64 wherein the unit dose contains 1.0 mg to 50 mg of escitalopram or a pharmaceutically acceptable salt thereof.

66. The method according to claim 64 wherein the unit dose is given once daily.

67. The method according to claim 66 wherein the daily doses are 5, 10 or 20 mg.

68. A method of facilitating psychotherapy, cognitive behavioural therapy or exposure-based therapy comprising administering a therapeutically effective amount of escitalopram or a pharmaceutically acceptable salt thereof.

69. The method according to claim 68 wherein the escitalopram or a pharmaceutically acceptable salt thereof is administered as a unit dose.

70. The method according to claim 69 wherein the unit dose contains 1.0 mg to 50 mg of escitalopram or a pharmaceutically acceptable salt thereof.

71. The method according to claim 69 wherein the unit dose is given once daily.

72. The method according to claim 71 wherein the daily doses are 5, 10 or 20 mg.
